(19) United States
(12) Patent Application Publication
(10) Pub. No.: US 2026/0041474 A1
(43) Pub. Date: Feb. 12, 2026

(54) CRYOABLATION PROBES AND RELATED METHODS

(71) Applicant: Varian Medical Systems, Inc., Palo Alto, CA (US)

(72) Inventors: Hongxuan Zhang, Austin, TX (US); Ryan Medema, Georgetown, TX (US); Vineel Vallapureddy, Plymouth, MN (US); Bryant Phamvu, Pflugerville, TX (US); Courtney Zimmel, Round Rock, TX (US)

(21) Appl. No.: 18/800,972

(22) Filed: Aug. 12, 2024

(51) Int. Cl. A61B 18/02; A61B 18/00; A61B 90/00

(52) U.S. Cl. CPC A61B 18/02; A61B 2018/00577; A61B 2018/00648; A61B 2018/00714; A61B 2018/00744; A61B 2018/00791; A61B 2018/0091; A61B 2018/0262; A61B 2018/0293; A61B 2090/064

(57) ABSTRACT

A cryoprobe includes a needle, and a handle comprising a distal portion aligned with and coupled to the needle and a proximate portion positioned adjacent to the distal portion. The proximate portion of the handle is oriented at an angle relative to the distal portion. The distal portion of the handle includes a first heat exchanger and the proximate portion of the handle includes a second heat exchanger

### FIELD

The present disclosure relates to cryoablation probes and related methods that include optimized heat exchange to account for pressure drops that may occur during transfer of cryogen.

### BACKGROUND

This section provides background information related to the present disclosure which is not necessarily prior art.

Systems and methods for providing cryoablation treatments may include cryoablation probes that are introduced at or near target tissue in a patient. A cryoablation system may include an extremely cold cryogen (liquid, gas, or mixed phase) that may be passed through a probe in thermal contact with the target tissue.

Heat from the tissue passes from the tissue, through the probe, and into the cryogen that removes heat from the targeted tissue. This removal of heat causes tissue to freeze, resulting in the destruction of the targeted tissue. It is desirable that the cryogen is of sufficiently low temperature to quickly and efficiently cause the targeted tissues to freeze.

Traditional or existing cryoprobes and related structures often include structures that fail to account for thermal energy transfer and/or pressure loss that may occur during transfer of the cryogen to the tip of the cryoprobe. Such existing cryoprobes and related structures may result in an inefficient use of cryogen and may take longer than needed to achieve sufficiently low temperatures at the target tissue. There exists a need, therefore, for improved cryoprobes and methods of use that efficiently achieve a desired temperature of the cryogen at the target tissue to produce iceballs in desired time periods.

### SUMMARY

This section provides a general summary of the disclosure, and is not a comprehensive disclosure of its full scope or all of its features.

In various embodiments of the present disclosure, a cryoablation apparatus and system is provided. The cryoablation system may be used to automatically adjust a flow of cryogen to a cryoprobe based on information obtained from one or more sensors positioned in or on the cryoprobe. The information may be collected in real-time during a freezing cycle to determine pressure drops that may occur in the cryoprobe. In some embodiments, the cryoprobe may include a first heat exchanger and a second heat exchanger positioned in the handle to lower a temperature of the supply cryogen using thermal energy transfer with returning cryogen. The cryoablation system may be configured to determine a pressure drop that may result of the thermal transfer between the supply cryogen and the return cryogen and to adjust the flow of cryogen according to maintain and/or achieve efficient freezing of a target tissue.

In various embodiments of the present disclosure, a cryoprobe is provided. The cryoprobe may include a needle, and a handle including a distal portion aligned with and coupled to the needle and a proximate portion positioned adjacent to the distal portion. The proximate portion may be oriented at an angle relative to the distal portion. The distal portion may include a first heat exchanger and the proximate portion includes a second heat exchanger. The proximate portion and the distal portion may each be positioned on different sides of a right-angle handle.

In one aspect, the needle may define a Joule-Thompson expansion chamber.

In another aspect, the first exchanger and the second heat exchanger may be separated from one another.

In another aspect, the cryoprobe may include a temperature sensor and a pressure sensor positioned in the handle between the first heat exchanger and the second heat exchanger.

In another aspect, the cryoprobe may include a first temperature sensor and a first pressure sensor located at a proximate end of the first heat exchanger, a second temperature sensor and a second pressure sensor located in the handle between the first heat exchanger and the second heat exchanger, and a third temperature sensor and a third pressure sensor located at a distal end of the second heat exchanger.

In another aspect, the angle may be 90 degrees.

In another aspect, the handle may be flexible to adjust the angle between the proximate portion and the distal portion.

In another aspect, the first heat exchanger and the second heat exchanger may be configured to cool supply cryogen using return cryogen flowing away from a tip of the needle.

In some embodiments of the present disclosure, a cryoablation system is provided. The cryoablation system may include one of the cryoprobes described herein, and a cryoablation control apparatus coupled to one or more sensors in the handle of the cryoprobe. The cryoablation control apparatus may include at least one processor and memory, and may be configured to obtain operating data from the one or more sensors and adjust at least one operating parameter of cryogen flowing in the cryoprobe.

In another aspect, the step of adjusting at least one operating parameter comprises changing a flow rate or pressure of supply cryogen flowing into the first heat exchanger based on a pressure drop of the supply cryogen flowing out of the first heat exchanger.

In another aspect, the one or more sensors in the handle may include a temperature sensor and a pressure sensor located in the handle between the first heat exchanger and the second heat exchanger.

In another aspect, the cryoablation control apparatus may be configured to maintain a temperature of a tip of the needle in a predetermined temperature range.

In another aspect, the cryoablation control apparatus may be further configured to minimize an amount of cryogen consumed during operation.

In another aspect, the step of adjusting the at least one operating parameter of the cryogen comprises modulating a flow rate of the cryogen.

In another aspect, one or more methods are provided. The method may include obtaining operating data from the one or more sensors in the needle of the cryoprobe of the present disclosure, and adjusting, by a cryoablation control apparatus comprising at least one processor and memory, at least one operating parameter of cryogen flowing in the cryoprobe.

In another aspect, the step of adjusting the at least one operating parameter may include changing a flow rate or pressure of supply cryogen flowing into the first heat exchanger based on a pressure drop of the supply cryogen flowing out of the first heat exchanger.

In another aspect, the one or more sensors in the handle may include a temperature sensor and a pressure sensor located in the handle between the first heat exchanger and the second heat exchanger.

In another aspect, the method may include maintaining a temperature of a tip of the needle in a predetermined temperature range.

In another aspect, the method may include minimizing an amount of cryogen consumed during operation.

In another aspect, the step of adjusting the at least one operating parameter of the cryogen may include modulating a flow rate of the cryogen.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an illustration of an example cryoablation apparatus in accordance with some embodiments of the present disclosure.

FIG. 2 is a side cross-sectional view of an example cryoprobe in accordance with some embodiments of the present disclosure.

FIG. 3 is a diagram of an example cryoablation system and a method of use in accordance with some embodiments of the present disclosure.

FIG. 4 is a Pressure-Enthalpy diagram illustrating aspects of the cryoprobes and methods of use in accordance with some embodiments of the present disclosure.

FIG. 5 is a diagram illustrating an example machine learning model that may be used in some embodiments of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms.

These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In some embodiments of the present disclosure, a cryoprobe is provided that includes a heat exchanger that is positioned and/or optimized to result in improved transfer of thermal energy from supply cryogen to return cryogen. The cryoprobes of the present disclosure may include a second heat exchanger spaced apart from a first heat exchanger to allow increased thermal energy to be transferred as compared to existing cryoprobe designs. The cryoprobes of the present disclosure may be optimized to account for temperature and/or pressure changes that may occur in the cryoprobe as a result of changes that may occur during operation of the cryoprobe during a freezing cycle. The cryoprobes of the present disclosure may be operated as part of a cryoablation system that may include a cryo-controller that may adjust operating parameters of the cryoprobe to improve and/or increase efficiency of the cryoprobe to optimize power used during a freezing cycle and/or cryogen usage.

The cryoprobes of the present disclosure may be configured to utilize the Joule-Thompson effect to significantly decrease the operating temperature of the tip of the cryoprobe. As explained above, the tip of the cryoprobe may be inserted at or near a target tissue during a cryoablation procedure. A cryogen is passed through the cryoprobe and allowed to remove heat from the target tissue. In some examples, Argon is used as the cryogen. In other examples, other cryogens may be used such as Nitrogen, or the like. During operation, the cryoprobe systems operate with the cryogen at extremely low temperatures. The target tissue at or near the tip of the cryoprobe freezes destroying the target tissue. It is desirable to freeze the target tissue quickly so as to reduce a likelihood of harm to healthy tissue and to reduce a length of cryoablation treatments.

In Joule-Thompson cryoprobes, the cryogen is transferred to the tip of the cryoprobe at a high pressure. In some instances, the pressure may be at or above 3000 pounds per square inch (psi). In other examples, the pressure may be at other elevated pressures. This high pressure cryogen is allowed to expand in a chamber in the tip of the cryoprobe whereby the drop in pressure is accompanied by a drop in temperature. The Joule-Thompson effect allows the temperature of the cryogen at the tip of the cryoprobe to drop to temperatures less than −100 degrees Celsius. It may be desirable to achieve even lower temperatures than −100 degrees Celsius such as temperatures lower than −120 degrees Celsius or −130 degrees Celsius.

In the clinical environment, a cryoablation treatment may be performed in a room in which the patient is positioned on a table or gantry that is moved into and out of an imaging device such as a Computed Tomography (CT) device or MRI device. The space in such imaging devices are limited. In an example treatment, the needle of the cryoprobe may be inserted into a patient at or near the target tissue in the patient. To confirm positioning of the needle and/or to collect other treatment information, the patient may be transferred into and out of the imaging device with the cryoprobes inserted at the target tissue. The cryoprobes may have a right angle bend at the handle to allow the cryogen supply/return lines to be routed in a manner to allow the patient to be moved into and out of the imaging device.

The cryoprobes of the present disclosure are improvements over existing cryoprobes because the cryoprobes of present disclosure may include improved or increased thermal transfer between the supply cryogen and the return cryogen. This, in turn, allows the cryogen to reach desired low temperatures to quickly produce an iceball to destroy the target tissue. In addition, the cryoprobes may be optimized to have a desired amount of thermal energy transfer to achieve the desired iceball size in a desired period of time. In addition, the cryoprobes of the present disclosure are improvements because the cryoprobes may achieve the desired conditions more quickly while consuming less cryogen. Due to the costs of the cryogen, this can lead to reduced operating costs.

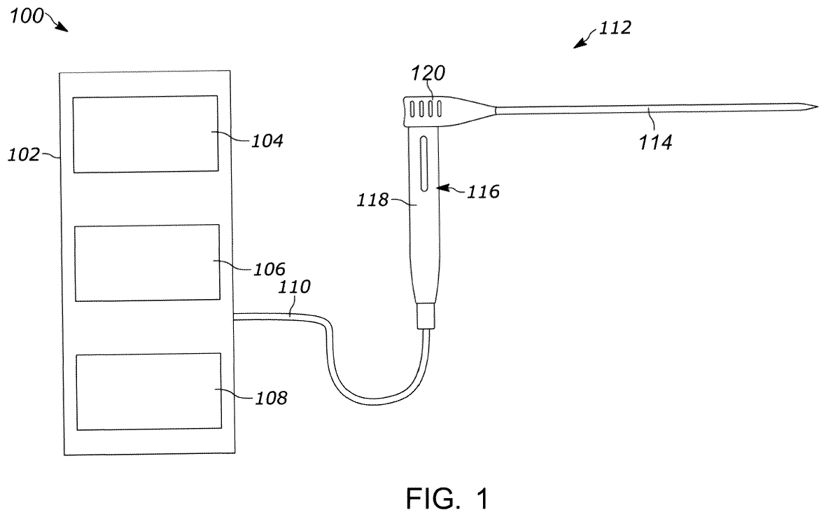

Referring now to FIG. 1, an example cryoablation apparatus 100 is shown. The cryoablation apparatus 100 may include a cryoablation console 102, and a cryoprobe 112. The cryoablation console 102 may include a cryo-controller 104, a cryogen delivery apparatus 106 and a cryogen source 108. While shown inside console 102 in FIG. 1, the cryogen source 108 may be located outside the console 102. The cryo-controller 104 may include a computing device or other controller that can be used to control delivery of a cryogen (e.g., Argon, Helium, Nitrogen, or the like) from the cryogen source 108 to the cryoprobe 112 using the cryogen delivery apparatus 106.

The cryogen source 108 may be a suitable Dewar or other container that can be filled with a cryogen. The cryogen delivery apparatus 106 may include a pump, one or more valves, and other suitable fluid delivery devices to fluidly connect the cryogen source to the cryogen line 110 of the cryoprobe 112. Upon the initiation of a freezing cycle, the cryo-controller 104 may cause the cryogen to be moved through a cryogen flow path that includes a cryogen supply line from the cryogen source through the cryogen line 110 to the cryoprobe 112. The cryogen may then flow back to the cryogen source via a cryogen return line from the cryoprobe 112 back to the cryogen source 108 or it may be vented to ambient air.

The cryogen line 110 may be a flexible tube or other conduit that may include multiple lumens to allow the cryogen to flow in a supply direction to the cryoprobe 114 and separately in a return direction away from the cryoprobe 114. The cryogen line 110 may of sufficient length to allow the console 102 to be positioned near the patient in a treatment room and to allow the patient to be moved into and out of an imaging device. In some examples, the cryogen line may be about 12 feet in length. In other examples, the cryogen line 110 may have other lengths. In other examples, the cryogen line 110 may be greater than about 12 feet. In other examples, the cryogen lien 110 may be less than about 12 feet in length.

The cryogen line 110 fluidly couples the cryogen source 108 to the cryoprobe 112. The cryoprobe 112 may include a needle 114. The needle 114 may be a pointed cylindrical tool or other elongated member that is configured to be inserted into patient tissue and be positioned at or near the target tissue during treatment. The needle 114 may be configured as a pointed tool having an outer diameter in a range of about 1 mm to about 4 mm. The cryoprobe 112 may also include a handle 116. The handle 116 may be configured with a first (or proximate) portion 118 and a second (or distal) portion 120. The first portion 118 may be substantially aligned with the cryogen line 110 and the second portion 120 may offset at an angle relative to the first portion 118. The offset angle between the first portion 118 and the second portion 119 may be about 90 degrees to define a right angle handle. In other example, the first portion 118 and the second portion 120 may be offset at different angles.

In some examples, the handle 116 may include a vacuum chamber positioned at or near an outside surface of the handle 116. The vacuum chamber may insulate the exterior of the handle from the extremely low operating temperatures of the cryogen that moves through the handle to the cryoprobe 112. This may allow an operator to touch or otherwise manipulate the cryoprobe 112 during treatment.

While not shown, more than one cryoprobe 112 may be coupled to the console 102. Multiple cryoprobes 112 may be used during a single cryoablation treatment in combination. The console 102 may be configured to deliver cryogen to each of the multiple cryoprobes 112. The cryoprobes 112 may be similar to each other or may be different to produce iceballs of different sizes and shapes so as to freeze and destroy the target tissue.

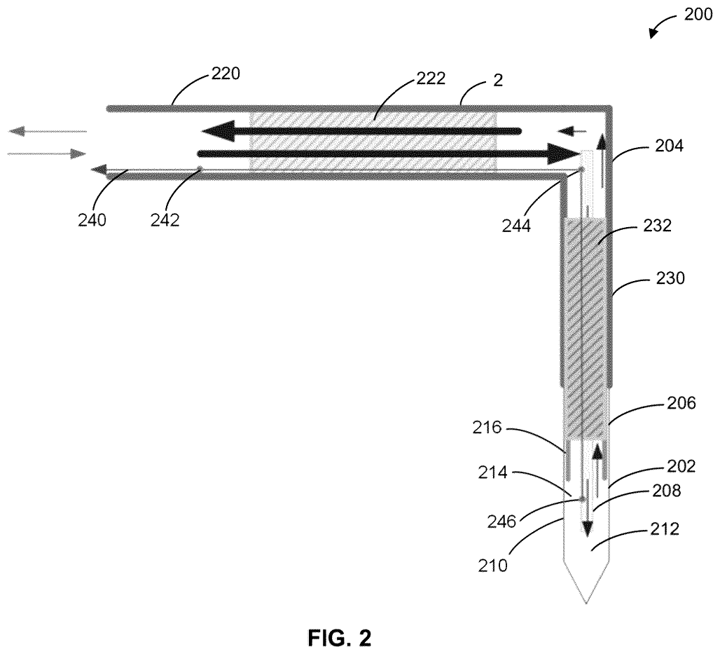

Referring now to FIG. 2, an example cryoprobe 200 is shown. The cryoprobe 200 may be used in combination with a cryoablation apparatus, such as the cryoablation apparatus 100 previously described. In this example, the cryoprobe 200 may include a needle 202 and a handle 204. The cryoprobe 200 may be coupled to a cryogen source using a cryogen line (not shown). The needle 202 may be any suitable cryoablation needle and may extend in an axial direction away from handle 204. The needle 202 may be made of suitable metal or alloy such as stainless steel. In other examples, other suitable materials such as ceramic may be used. The needle 202 may have various sizes or diameters that may be suitable to produce a freezing zone or iceball that may be desired to freeze the target tissue. In various examples, the needle 202 may have an outer diameter in a range of about 1 mm to about 4 mm. In other examples, other suitable sizes may be used.

The needle 202 may include an outer shell 206 and an inner lumen 208 that extends radially along its length. The outer shell 206 and the inner lumen 208 may be positioned concentrically with each other. The inner lumen 208 may include an opening at one end toward a tip 210 of the needle. During a freezing cycle, the cryogen (at high pressure) may be passed through the inner lumen 208 toward the tip 210 and the cryogen may exit the inner lumen 208 and allowed to expand in a Joule-Thompson chamber 212 at the tip 210. Such expansion causes the temperature of the cryogen to drop. The cryogen may then flow away from the tip 210 through a return pathway 214 defined as a space between an inner surface of the outer shell 206 and the outer surface of the inner lumen 208.

The needle 202 may also include a vacuum sleeve 216. The vacuum sleeve 216 may be a cylindrical sleeve enclosing a space sealed at vacuum. The vacuum sleeve 216 may insulate the outer shell 206 from the cryogen that is passing through the needle 202. The vacuum sleeve 216 may be positioned relative to the tip 210 to determine a portion of the needle 202 located at the tip 210 that achieves the low temperature of the cryogen to create an iceball. The position of the vacuum sleeve 216 relative to the tip 210 may determine a size of the iceball that is formed during operation of the cryoprobe 200.

The needle 202 may be coupled to the handle 204. The handle 204 may be formed of plastic, composite, or other suitable material. The handle 204 may be fluidly and mechanically coupled to the needle 202. The inner lumen 208 and the outer shell 206 of the needle 202 may be fluidly coupled to a supply lumen and a return pathway, respectively. In this manner, the supply cryogen may be supplied to the needle through the handle 204 and the return cryogen may flow from the needle 202 to the cryogen line. The handle 204 may also include a vacuum chamber. The vacuum chamber may be positioned at an outer side of the handle 204 and insulate an outer surface of the handle 204 from the extreme temperatures of the cryogen.

In the example shown, the handle 204 is a right-angle handle that transitions from a longitudinal or axial direction of the cryogen line (not shown) to a longitudinal or axial direction of the needle 202. In this example, the needle 202 is oriented at 90 degrees or perpendicular to the axial direction of the cryogen line. As shown, the handle 204 includes a first portion 220 and a second portion 230. For purposes of the present disclosure, the first portion 220 may alternatively be called the proximal portion 220 for being located proximal the cryoablation console during operation. The second portion 230 may alternatively be called the distal portion 230 for being located distal the cryoablation console during operation. In this example, the distal portion 230 is oriented perpendicularly from the proximal portion 220. In other examples, the portions of the handle 204 may be oriented at a different angle from each other. In still other examples, the handle 204 may be flexible to allow adjustment of the relative orientation of portions of the handle 204.

The handle 204 of cryoprobe 200 may include a first heat exchanger The heat exchanger is used to transfer thermal energy from supply cryogen that is entering the cryoprobe 200 to return cryogen that flowing away from the needle 202. This heat transfer may lower a temperature of the incoming supply cryogen.

The handle 204 may include a first heat exchanger 222 and a second heat exchanger 232. The first heat exchanger 222 may be positioned in the proximal portion 220 of the handle 204 and the second heat exchanger 232 may be positioned in the distal portion 230 of the handle 204. The first heat exchanger 222 and the second heat exchanger 232 may be separated or spaced apart from one another. The first heat exchanger 222 and the second heat exchanger 232 may be separated from one another by a corner of the handle 204.

The first heat exchanger 222 and the second heat exchanger 232 may have similar structures or may have different structures. The heat exchangers 222, 232 may, for example, be configured as counter-flow heat exchangers. The heat exchangers 222, 232 may include fins, ribs, helical coils, or other non-planar surfaces to increase a surface area through which thermal energy may be transferred between the supply cryogen and the return cryogen. In some examples, the supply cryogen may flow through an internal flow path and the return cryogen may flow through an outer flow path over the inner flow path of the supply cryogen. The material of the inner lumen or internal flow path may be made of a material with a coefficient of thermal transfer such as copper or brass. In other examples, other structures and other materials may be used.

The proximal heat exchanger 222 and the distal heat exchanger 232 operate to lower a temperature of the supply cryogen before the supply cryogen expands in the Joule-Thompson expansion chamber of the needle 202. It may be desirable to achieve temperatures of at least −120 degrees Celsius at the tip of the needle 202 to quickly form an iceball at the tip 210 of the needle 202. To achieve this low temperature, the temperature of the supply cryogen is lowered from the incoming temperature of the supply cryogen when it enters the handle 204 of the cryoprobe 200. Without lowering of the temperature of the cryogen in the handle 204, the temperatures of the cryogen do not reach the desired cryoablation temperature threshold (e.g., −120 degrees Celsius or lower).

The proximal heat exchanger 222 and the distal heat exchanger 232 allow the temperature of the supply cryogen to be lowered in both the proximal portion 220 and in the distal portion 230 of the handle 204. It has been observed that in configurations in which the handle 204 includes a proximal heat exchanger 222 (and not a distal heat exchanger 232), the cryogen experiences a pressure drop as the temperature of the cryogen drops in the proximal heat exchanger 222. In one example, Argon was used as a cryogen and the pressure was observed to drop in a range of about 10-25% after the cryogen passed through the proximal heat exchanger 222. If such cryogen is not further cooled, the reduced pressure of the cryogen may not be high enough to achieved the desired low temperatures at the tip 210 of the needle 202 after expansion. Thus, the handle 204 includes a second heat exchanger 232 to further cool the supply cryogen prior to the supply cryogen reaching the tip 210 of the needle 202.

As further shown in FIG. 2, the cryoprobe 200 may also include a signal line 240. The signal line 240 may be one or more wires included in or on the cryoprobe 200 to provide signals to the cryo-controller of one or more sensors in the cryoprobe 200. In one example, the cryoprobe 200 may include a first sensor point 242 that may include one or more sensors to determine a temperature and pressure of the supply cryogen at a location incoming to the handle 204. The cryoprobe 200 may also include a second sensor point 244. The second sensor point 244 may include one or more sensors to determine a temperature and pressure of the supply cryogen at a location at the corner of the handle 204 and/or between the proximal heat exchanger 222 and the second heat exchanger 232. The cryoprobe 200 may also include a third sensor point 246. The third sensor point 246 may be located at or near the tip 210 of the needle 202. The third sensor point 246 may be configured to provide a temperature and pressure of the supply cryogen at a location downstream of the distal heat exchanger 232 at or near the tip 210 of the needle 202. Each of the first sensor point 242, the second sensor point 244, and the third sensor point may be coupled to the signal line 240 to provide information regarding the temperature and pressure at the sensor point locations.

The cryo-controller may be coupled to the signal line 240. The cryo-controller may operate to make adjustments to the operation of the cryoprobe 200 in response to receiving information from the first sensor point 242, the second sensor point 244, and/or the third sensor point 246. For example, the cryo-controller may determine that a pressure drop has occurred in the proximate heat exchanger 222 by comparing a temperature and/or pressure from the second sensor point 244 to the temperature and/or pressure from the first sensor point 242. The cryo-controller may increase a pressure of the supply cryogen a result of this determination. The cryo-controller may make similar or other adjustments to the flow of cryogen after comparing a temperature and/or pressure of cryogen at the third sensor point 246 to the information from the second sensor point 244 and/or from the first sensor point 242.

In other examples, the information from the first sensor point 242, the second sensor point 244 and/or the third sensor point 246 may be used to optimize and/or design a desired configuration of the cryoprobe 200. Upon learning a pressure drop across the proximate heat exchanger 222, the design of the cryoprobe 200 may be configured to size the distal heat exchanger 232 to the size needed to further drop the temperature of the cryogen to achieve the desired cryoablation temperature levels. It may be desirable, for example, to minimize a size of the distal heat exchanger 232 so as to not unnecessarily grow the size of the distal portion 230 of the handle 204. In other circumstances, the information from the sensor points may be used to optimize the size of both the proximate heat exchanger 222 and the second heat exchanger 232 to minimize and/or optimize a size of the handle 204. As such, the cryoprobes of the present disclosure may be efficiently designed to maintain lower costs over those of other designs.

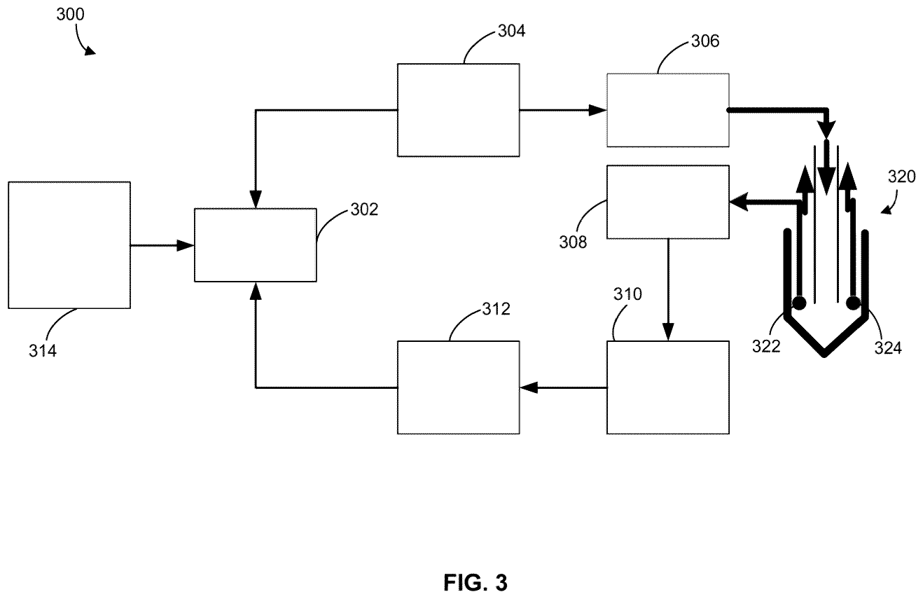

Referring now to FIG. 3, an example cryoablation system 300 is shown. The cryoablation system 300 may incorporate the aspects or elements previously described such as the cryoprobe 200. In this example, the cryoablation system 300 may include a cryo-controller 302 that is coupled to a cryoprobe 320. The cryoprobe 320 may be similar to the cryoprobe 200 previously described. The cryoprobe 320 may include sensor points 322, 324 that may collect information regarding the temperature and pressure of the cryogen in the cryoprobe 320. While only two sensor points 322, 324 are shown, the cryoprobe 320 may include multiple sensor points located throughout or along a cryogen path to measure the temperature, pressure and/or other characteristics (e.g., flow rate, impedance, etc.). For example, multiple sensor points may be located at or between multiple heat exchangers located in the handle of the cryoprobe 320.

The information collected from the sensor points 322 may be collected by a cryoprobe collector 308. The cryoprobe collector 308 may be a data acquisition unit, bus, or other suitable device to collect signals from the sensors 322, 324. The cryoprobe collector 308 maybe operatively connected to the sensor points 322, 324 using a suitable wired or wireless connection.

The information from the sensor points 322, 324 may be provided to an information conditioner 310. The information conditioner 310 may be suitable software or hardware that is configured to process the information so that it may be used by the cryoablation system 300. The information conditioner 310 may, for example, digitize, filter, condition, de-noise, or perform other transformations to the information or signals from the sensor points 322, 324. The information conditioner 310 may also provide the processed information to a comparator 312.

The comparator 312 may be a software module or other suitable hardware that may compare the information from the sensor points 322 to treatment parameters, operating thresholds, predetermined operating ranges, treatment plans, or other information to determine whether further action is needed to implement changes to the operation of the cryoablation system to improve efficiency or other operating performance of the cryoablation system 300.

The cryoablation system 300 may also include a cryo-model 304. The cryo-model 304 may be, for example, a trained machine learning model as will be further described below. The cryo-model 304 may operate to receive inputs such as information regarding the operating conditions of the cryoprobe 320 from the sensor points 322, 324 and determine recommended outputs such as a preferred cryogen temperature, pressure, flow rate, flow rate modulation, or the like.

The cryo-controller 302 and/or the cryo-model 304 may be coupled to a cryogen regulator 306. The cryogen regulator 306 may include one or more device that can control the flow of cryogen in the cryoprobe 320. The cryogen regulator 306 (while not shown) may be coupled to a source of cryogen such as a Dewar or the like. The cryogen regulator 306 may include one or more electrically controlled valves that can be opened or closed to start, stop, or modify a flow of cryogen to the cryoprobe 320. The cryogen regulator 306 may also include one or more pumps that may be used to start, stop or modify a flow of cryogen to the cryoprobe 320. Such pumps may also be used to change a pressure of the cryogen being supplied to the cryoprobe 320. In other examples, the cryogen regulator 306 and a pump included therein may be used to modulate the flow of cryogen. Such modulation may cause pulses or intermittent flow of cryogen to be provided with varying levels of temperature, pressure, flow rates, or other varying flow characteristics.

The cryo-controller 302 may also be coupled to a treatment database 314. The treatment database may include various types or sources of information that may include clinical or treatment procedures, patient information, or other information regarding that may be used during a cryoablation treatment. The information may also include tissue information, iceball formation information, desired operating thresholds, desired operating ranges, and the like. This information may be accessed and used by the cryo-controller 302.

The cryo-controller 302 may operate to control and adjust the operation of the cryoprobe 320 during a cryoablation treatment. Such a cryoablation treatment may include a freezing cycle which includes the creation of an iceball at the target tissue in the patient. In one example method, the cryo-controller 302 may operate with an automatic and adaptive closed-loop control based on the information received from the sensor points 322, 324 in or on the cryoprobe 320. In such methods, the sensor points 322, 324 may obtain temperature and pressure measurements in real-time during a freezing cycle and relay such information via the cryoprobe collector 308, the information conditioner 310, and/or the comparator 312 to the cryo-controller 302. The cryo-controller 302, using the cryo-model 304, may adjust or modify the flow of cryogen to the cryoprobe 320 using the cryogen regulator 306.

In one example, the sensor points may provide information regarding a pressure drop and/or temperature change across a heat exchanger that may be positioned in the handle of the cryoprobe 320. In response and based on the information regarding the temperature change and/or a pressure drop after the heat exchanger, the cryo-controller 302 may raise the pressure or lower the temperature of the cryogen flowing to the cryoprobe 320. Such a change may be needed to achieve a desired temperature at the tip of the cryoprobe 320. In other examples, other changes or other adjustments may be automatically made during operating of the cryoprobe 320.

The cryoablation system 300 illustrates the cryo-controller 302, the cryo-model 304, the cryoprobe collector 308, the information conditioner 310, and the comparator 312 as separate elements. It should be appreciated that the cryo-controller 302, the cryo-model 304, the cryoprobe collector 308, the information conditioner 310, and the comparator 312 may be combined into a single piece of hardware or various individual elements may be incorporated or separated into individual modules or devices. The cryo-controller 302 may be a suitable controller, computer, PLC, application specific circuit, smart device, computing device, or the like. The cryo-controller 302 may include a process and memory that may include individual modules that include executable instructions that may perform the functionality described herein. The elements of cryoablation system 300 may combined into a common computing and/or may be coupled using a suitable wired or wireless connection.

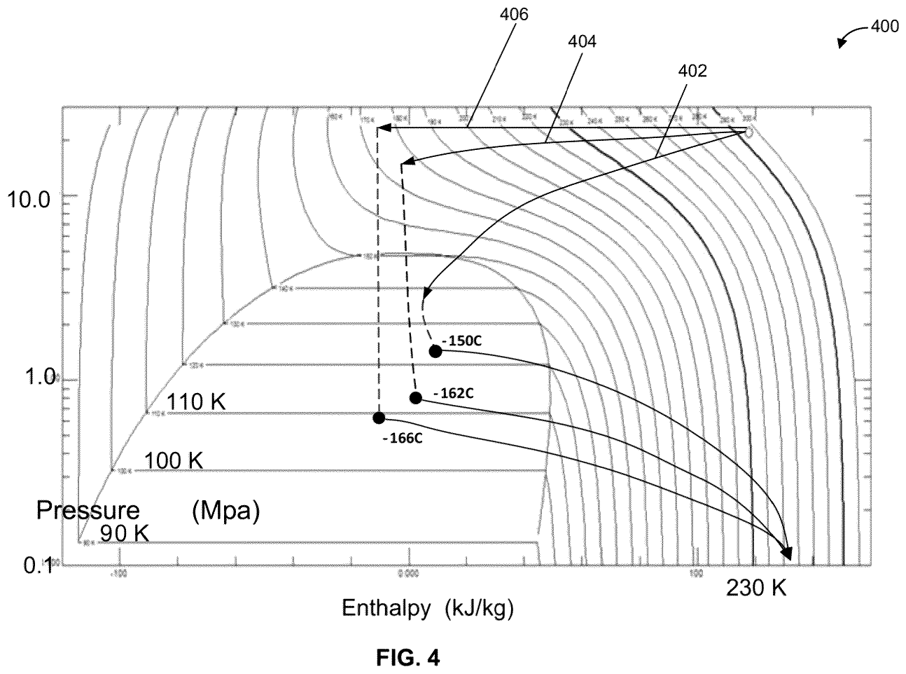

As shown in FIG. 4, operating efficiencies may be obtained using the cryoablation systems, apparatuses and cryoprobes of the present disclosure. FIG. 4 provides a Pressure-Enthalpy chart 400 for an example cryogen, in this case Argon.

The curve 402 illustrates a curve that represents the thermodynamic cycle of the Argon during a freezing cycle in an existing cryoprobe. The existing cryoprobe does not include the optimized closed loop control processes previously described. The curve 406 illustrates an example thermodynamic cycle for Argon in an ideal case without any pressure or thermal loss of the cryogen as it travels through the cryoablation apparatus and cryoprobe. The curve 404 represents an optimized thermodynamic cycle for Argon that may be achieved using the cryoprobes of the present disclosure and the automatic closed-loop adjustment process described herein. As can be seen, by using the cryoprobes of the present disclosure and the closed loop adjustment system and process, the cryogen may achieve a temperature of at or around −162 degrees Celsius which is significantly lower than the −150 degrees Celsius temperature achieved without the automatic control.

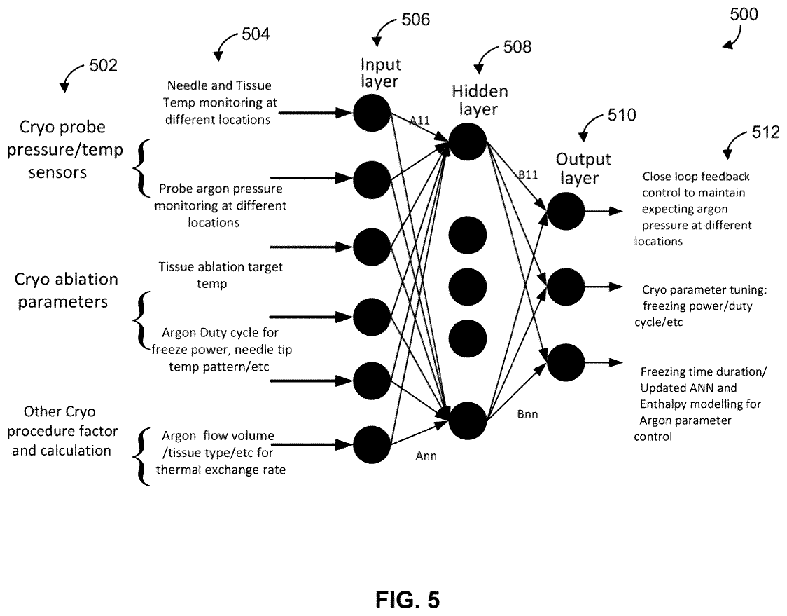

Referring now to FIG. 5, an example cryo-model 500 is shown. The cryo-model 500 is one example that may be used in the cryoablation system 300 previously described. The cryo-model 500 may be a trained machine learning model that may use an artificial neural network to determine complex and non-linear relationships between inputs. The cryo-model 500 may be created using an open-source or proprietary artificial neural network tool or framework. The cryo-model 500 may include an input layer 506, a hidden layer 508, and an output layer 510. The input layers may include various types of information that may be measured or obtained regarding a cryoablation treatment or a freezing cycle. The artificial neural network may include one or more hidden layers 508 that may identify relationships between the inputs and the outputs.

In one example, input sources 502 may provide input information 504 to the cryo-model 500. The input sources 502 may include various pieces of information regarding a cryoablation or freezing cycle. The information may include pressure information or temperature information regarding the cryogen that may be obtained from one or more sensors in the cryoprobe or elsewhere in the cryoablation apparatus.

Other information may include treatment parameters such as target temperatures, tissue characteristics, duty cycle information, a tip temperature pattern, flow rate, flow volume, and/or thermal exchange rate. Such information may be obtained from sensors or from a clinical procedures, treatment plans or the like. All such information may be used as an input 504 and provided to the input layer 506 of the cryo-model 500. After processing using the one or more hidden layers 508, the cryo-model 500 may output various pieces of information that may be used by the cryo-controller.

The outputs 512 of the cryo-model 500 may include recommendations for pressure levels, duty cycles, cryogen flow, freezing time duration or the like. The cryo-controller 302 may implement such changes in real-time during a freezing cycle to obtain optimized or improvements in efficiency.

The cryo-model 500 may be trained before it is implemented in the cryoablation system 300. The cryo-model 500 may be trained using a training set of data that may include various input information such as that described above. The cryo-model may be trained using a suitable loss function or cost function. In some examples, the cryo-model 500 may be trained to minimize an amount of cryogen consumed in some examples. In other examples, the cryo-model 500 may be trained to achieve a lowest output temperature at the cryoprobe. In other examples, the cryo-model 500 may be trained using a function that is a combination of various performance measures that may include cryogen consumed, energy consumed, time to achieve iceball size, temperature at cryoprobe, cost, etc.

The term model as used in the present disclosure includes data models created using machine learning and/or artificial intelligence. Machine learning may involve training a mathematical model in a supervised or unsupervised setting. Machine learning models may be trained to learn relationships between various groups of data. The models may be based on a set of algorithms that are designed to model abstractions in data by using a number of processing layers. The processing layers may be made up of non-linear transformations. Machine learning models may include, for example, neural networks, convolutional neural networks and deep neural networks. Such neural networks may be made of up of levels of trainable filters, transformations, projections, hashing, and pooling. The models may be used in large-scale relationship-recognition tasks. The models can be created by using various open-source and proprietary machine learning tools and/or libraries known to those of ordinary skill in the art.

The following is a list of non-limiting illustrative embodiments disclosed herein:

Illustrative embodiment 1: A cryoprobe comprising a needle; and a handle comprising a distal portion aligned with and coupled to the needle and a proximate portion positioned adjacent to the distal portion, the proximate portion oriented at an angle relative to the distal portion, wherein the distal portion includes a first heat exchanger and the proximate portion includes a second heat exchanger.

Illustrative embodiment 2: The cryoprobe of illustrative embodiment 1, wherein the needle defines a Joule-Thompson expansion chamber.

Illustrative embodiment 3: The cryoprobe of any of illustrative embodiments 1 or 2, wherein the first exchanger and the second heat exchanger are separated from one another.

Illustrative embodiment 4. The cryoprobe of any of illustrative embodiments 1 to 3, comprising a temperature sensor and a pressure sensor positioned in the handle between the first heat exchanger and the second heat exchanger.

Illustrative embodiment 5. The cryoprobe of any of illustrative embodiments 1 to 4, comprising: a first temperature sensor and a first pressure sensor located at a proximate end of the first heat exchanger; a second temperature sensor and a second pressure sensor located in the handle between the first heat exchanger and the second heat exchanger; and a third temperature sensor and a third pressure sensor located at a distal end of the second heat exchanger.

Illustrative embodiment 6. The cryoprobe of any of illustrative embodiments 1 to 5, wherein the angle is 90 degrees.

Illustrative embodiment 7. The cryoprobe of any of illustrative embodiments 1 to 5, wherein the handle is flexible to adjust the angle between the proximate portion and the distal portion.

Illustrative embodiment 8. The cryoprobe of any of illustrative embodiments 1 to 7, wherein the first heat exchanger and the second heat exchanger are configured to cool supply cryogen using return cryogen flowing away from a tip of the needle.

Illustrative embodiment 9. A cryoablation system comprising:

the cryoprobe of any of illustrative embodiments 1 to 8;

a cryoablation control apparatus coupled to one or more sensors in the handle of the cryoprobe, the cryoablation control apparatus comprising at least one processor and memory, the cryoablation control apparatus configured to:

obtain operating data from the one or more sensors; and adjust at least one operating parameter of cryogen flowing in the cryoprobe.

Illustrative embodiment 10. The cryoablation system of illustrative embodiment 9, wherein the step of adjusting at least one operating parameter comprises changing a flow rate or pressure of supply cryogen flowing into the first heat exchanger based on a pressure drop of the supply cryogen flowing out of the first heat exchanger.

Illustrative embodiment 11. The cryoablation system of any of illustrative embodiments 9 or 10, wherein the one or more sensors in the handle comprises a temperature sensor and a pressure sensor located in the handle between the first heat exchanger and the second heat exchanger.

Illustrative embodiment 12. The cryoablation system of any of illustrative embodiments 9 to 11, wherein the cryoablation control apparatus is configured to maintain a temperature of a tip of the needle in a predetermined temperature range.

Illustrative embodiment 13. The cryoablation system of any of illustrative embodiments 9 to 12, wherein the cryoablation control apparatus is further configured to minimize an amount of cryogen consumed during operation.

Illustrative embodiment 14. The cryoablation system of any of illustrative embodiments 9 to 13, wherein the step of adjusting the at least one operating parameter of the cryogen comprises modulating a flow rate of the cryogen.

Illustrative embodiment 15. A method comprising: obtaining operating data from the one or more sensors in the needle of the cryoprobe of illustrative embodiment 1; and adjusting, by a cryoablation control apparatus comprising at least one processor and memory, at least one operating parameter of cryogen flowing in the cryoprobe.

Illustrative embodiment 16. The method of illustrative embodiment 15, wherein the step of adjusting the at least one operating parameter comprises changing a flow rate or pressure of supply cryogen flowing into the first heat exchanger based on a pressure drop of the supply cryogen flowing out of the first heat exchanger.

Illustrative embodiment 17. The method of any of illustrative embodiments 15 or 16, wherein the one or more sensors in the handle comprises a temperature sensor and a pressure sensor located in the handle between the first heat exchanger and the second heat exchanger.

Illustrative embodiment 18. The method of any of illustrative embodiments 15 to 17, further comprising maintaining a temperature of a tip of the needle in a predetermined temperature range.

Illustrative embodiment 19. The method of any of illustrative embodiments 15 to 18, further comprising minimizing an amount of cryogen consumed during operation.

Illustrative embodiment 20. The method of any of illustrative embodiments 15 to 20, wherein the step of adjusting the at least one operating parameter of the cryogen comprises modulating a flow rate of the cryogen.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A cryoprobe comprising:
a needle; and
a handle comprising a distal portion aligned with and coupled to the needle and a proximate portion positioned adjacent to the distal portion, the proximate portion oriented at an angle relative to the distal portion,
wherein the distal portion includes a first heat exchanger and the proximate portion includes a second heat exchanger.

2. The cryoprobe of claim 1, wherein the needle defines a Joule-Thompson expansion chamber.

3. The cryoprobe of claim 1, wherein the first exchanger and the second heat exchanger are separated from one another.

4. The cryoprobe of claim 1, comprising a temperature sensor and a pressure sensor positioned in the handle between the first heat exchanger and the second heat exchanger.

5. The cryoprobe of claim 1, comprising:
a first temperature sensor and a first pressure sensor located at a proximate end of the first heat exchanger;
a second temperature sensor and a second pressure sensor located in the handle between the first heat exchanger and the second heat exchanger; and
a third temperature sensor and a third pressure sensor located at a distal end of the second heat exchanger.

6. The cryoprobe of claim 1, wherein the angle is 90 degrees.

7. The cryoprobe of claim 1, wherein the handle is flexible to adjust the angle between the proximate portion and the distal portion.

8. The cryoprobe of claim 1, wherein the first heat exchanger and the second heat exchanger are configured to cool supply cryogen using return cryogen flowing away from a tip of the needle.

9. A cryoablation system comprising:
the cryoprobe of claim 1;
a cryoablation control apparatus coupled to one or more sensors in the handle of the cryoprobe, the cryoablation control apparatus comprising at least one processor and memory, the cryoablation control apparatus configured to:
obtain operating data from the one or more sensors; and
adjust at least one operating parameter of cryogen flowing in the cryoprobe.

10. The cryoablation system of claim 9, wherein the step of adjusting at least one operating parameter comprises changing a flow rate or pressure of supply cryogen flowing into the first heat exchanger based on a pressure drop of the supply cryogen flowing out of the first heat exchanger.

11. The cryoablation system of claim 9, wherein the one or more sensors in the handle comprises a temperature sensor and a pressure sensor located in the handle between the first heat exchanger and the second heat exchanger.

12. The cryoablation system of claim 9, wherein the cryoablation control apparatus is configured to maintain a temperature of a tip of the needle in a predetermined temperature range.

13. The cryoablation system of claim 12, wherein the cryoablation control apparatus is further configured to minimize an amount of cryogen consumed during operation.

14. The cryoablation system of claim 9, wherein the step of adjusting the at least one operating parameter of the cryogen comprises modulating a flow rate of the cryogen.

15. A method comprising:
obtaining operating data from the one or more sensors in the needle of the cryoprobe of claim 1; and
adjusting, by a cryoablation control apparatus comprising at least one processor and memory, at least one operating parameter of cryogen flowing in the cryoprobe.

16. The method of claim 15, wherein the step of adjusting the at least one operating parameter comprises changing a flow rate or pressure of supply cryogen flowing into the first heat exchanger based on a pressure drop of the supply cryogen flowing out of the first heat exchanger.

17. The method of claim 15, wherein the one or more sensors in the handle comprises a temperature sensor and a pressure sensor located in the handle between the first heat exchanger and the second heat exchanger.

18. The method of claim 15, further comprising maintaining a temperature of a tip of the needle in a predetermined temperature range.

19. The method of claim 18, further comprising minimizing an amount of cryogen consumed during operation.

20. The method of claim 15, wherein the step of adjusting the at least one operating parameter of the cryogen comprises modulating a flow rate of the cryogen.
